SEQUENCE LISTING (2 sequences)

SEQ ID NO: 1 - protein, 887 aa, Homo sapiens
MGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 426 aa, synthetic construct
EESPFVSNPGNITGARGLTGTLRCQLQVQGEPPEVHWLRDGQILELVDSTQTQVPLGEDEQGDWIVASQLRITSLQLSDTGQYQCLVFLGHQTFVSQPGYVRLEGLPYFLEEPEDRTVAANTPFNLSCQAQGPPEPVDLLWLQDAVPLATAPGHGPQRSLHVPGLNKTSSFSCEAHNAKGVTTSRTATITVLPQQGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2024/0374685 A1
(43) Pub. Date: Nov. 14, 2024

(54) METHODS OF TREATING LOCALLY ADVANCED OR METASTATIC PANCREATIC ADENOCARCINOMA USING AXL DECOY RECEPTORS AS FIRST-LINE THERAPY

(71) Applicant: Aravive Inc, Houston, TX (US)

(72) Inventors: Gail McIntyre, Lansdale, PA (US); Reshma Rangwala, Philadelphia, PA (US)

(73) Assignee: Aravive Inc, Houston, TX (US)

(21) Appl. No.: 18/690,722

(22) PCT Filed: Sep. 12, 2022
(86) PCT No.: PCT/US2022/043234

§ 371 Date: Mar. 9, 2024

Related U.S. Application Data
(60) Provisional application No. 63/243,113, filed on Sep. 11, 2021.

(51) Int. Cl. A61K 38/17; A61K 31/337; A61K 31/7068; A61P 35/00

(52) U.S. Cl. CPC A61K 38/179; A61K 31/337; A61K 31/7068; A61P 35/00

(57) ABSTRACT

Compositions and methods are provided for the treatment of pancreatic ductal adenocarcinoma in a human patient, comprising the administration of a therapeutic dose of soluble AXL variant polypeptide as monotherapy or in combination with nab-paclitaxel and gemcitabine as first-line therapy according to a regimen determined to achieve stable disease/response (e.g., overall response rate (ORR)), longer PFS, and OS as compared to control.

### RELATED APPLICATIONS

This application claims benefit of U.S. Provisional Application No. 63/243,113, filed on Sep. 11, 2021, incorporated in its entirety by reference herein.

### STATEMENT REGARDING FEDERALLY SPONSORED RESEARCH OR DEVELOPMENT

This work was supported by Cancer Prevention & Research Institute of Texas, New Company Product Development Award DP150127. The State of Texas, USA, may have rights in any patent issuing on this application.

### SEQUENCE LISTING

The instant application contains a Sequence Listing in the form of a “paper copy” (PDF File) and a file containing the referenced sequences (SEQ D NOS: 1 and 2) in computer readable form (ST26 format text file) which is submitted herein. The Sequence Listing is shown using standard three letter code for amino acids, as defined in 37 C.F.R. 1.822.

### TECHNICAL FIELD

Pancreatic ductal adenocarcinoma (PDAC) is the twelfth most common cancer in the United States (US). The median age at diagnosis is 70 years and almost 90% of cases occur after the age of 55 years. In 2021 in the US, an estimated 60,430 people will be diagnosed with pancreatic cancer and more than 48,220 people will die from the disease (American Cancer Society. Cancer Facts and Figures, 2021). Pancreatic cancer has the highest mortality rate of all major cancers. It is currently the third leading cause of cancer-related death in the US after lung and colon cancers. The 5-year survival rate for localized, regional, distant disease, and all stages is 29%, 11%, 3%, and 8% respectively.

Surgery, radiation therapy and chemotherapy are treatment options that extend survival or relieve symptoms, but seldom lead to a cure. Surgical removal of the tumor is possible in less than 20% of patients diagnosed with pancreatic cancer given that the disease has often spread beyond the pancreas at the time of diagnosis. Adjuvant treatment with chemotherapy (and sometimes radiation) may lower the risk of recurrence. For advanced disease, chemotherapy (sometimes in combination with targeted drug therapies) may lengthen survival.

Targeted therapies and immunotherapies have not yet been fully integrated into pancreatic treatment regimens. While actionable mutations have identified in pancreatic adenocarcinomas, these mutations are exceedingly rare and associated treatments limited to small subsets of patients.

For the vast majority of pancreatic adenocarcinoma patients, systemic treatment with chemotherapy, including nab-paclitaxel in combination with gemcitabine is the standard of care. Per National Comprehensive Cancer Network guidelines, patients with metastatic disease who have an Eastern Cooperative Oncology Group performance status (ECOG-PS) of 0 or 1 should be considered for nab-paclitaxel and gemcitabine, FOLFIRINOX, or modified FOLFIRINOX as first-line (1L) treatment (National Comprehensive Cancer Network Guidelines, Pancreatic Adenocarcinoma, Version 2.2021).

In a Phase 1/2 trial of nab-paclitaxel (at 100, 125, and 150 mg/m2) plus gemcitabine in 67 subjects with advanced pancreatic cancer, the PR rate was 48%, with an additional 20% of patients demonstrating stable disease (SD) for 16 or more weeks, in the group of subjects treated with nab-paclitaxel at 125 mg/m2, the median PFS was 7.9 months (95% CI, 5.8 to 11.0). Median overall survival (OS) was 12.2 months (95% CI, 8.9 to 17.9). Dose limiting toxicities observed at 150 mg/m2 were sepsis and neutropenia in 1 subject, and grade 3 AE of fatigue and leukopenia in 2 subjects. The most common treatment-related adverse events (AEs) of any grade were anemia (98%), leukopenia (91%), neutropenia (89%), thrombocytopenia (83%), fatigue (76%), alopecia (76%), sensory neuropathy (63%), and nausea (48%). Most of these treatment-related AEs were grade 1 and 2. Specifically, ≥grade 3 nonhematologic AEs attributed to nab-paclitaxel-related were fatigue (21%) and sensory neuropathy (15%). Of the grade ≥3 treatment-related hematologic AEs, neutropenia (67%), leukopenia (44%), and thrombocytopenia (23%) were the most common (Von Hoff, D., J Clin Oncology., 29:4548-4554, 2011).

Based on these results a randomized, open-label phase III trial was performed to evaluate the efficacy and safety of gemcitabine vs gemcitabine+nab-paclitaxel as a 1L treatment in patients with metastatic pancreatic adenocarcinoma. In total, 861 subjects were randomized. The trial met its primary endpoint with an OS of 8.5 months for the combination vs. 6.7 months for gemcitabine alone (HR, 0.72; P<0.0001). OS was associated with a decrease in carbohydrate antigen 19-9 (CA 19-9) levels. Subjects with a >50% decrease in CA 19-9 levels had a 62% objective response rate (ORR) and OS of 13.6 months, whereas those with <50% decrease in CA 19-9 level had a 33% ORR and OS of 6.5 months. The median progression-free survival was 5.5 months in the nab-paclitaxel-gemcitabine group, as compared with 3.7 months in the gemcitabine group (hazard ratio for disease progression or death, 0.69 (95% CI, 0.58 to 0.82; P<0.001), and the response rate according to independent review was 23% versus 7% (P<0.001).

The most frequently reported nonhematologic AEs related to the combination were fatigue (54%), alopecia (50%), and nausea (49%). The incidence of peripheral neuropathy leading to the discontinuation of nab-paclitaxel was 8%, and the incidence leading to a dose reduction was 10%. The proportion of patients with SAEs was similar in the two treatment groups (50% with nab-paclitaxel+gemcitabine and 43% with gemcitabine alone). Fatal events were reported for 4% of the subjects in each treatment group. Sepsis (all grades) was reported more often in the nab-paclitaxel plus gemcitabine group than in the gemcitabine group (5% vs. 2%), as was pneumonitis (4% vs. 1%) (Von Hoff, D., New England Journal of Medicine, 369:1691-1703, 2013).

In spite of recent advances, there still exists an unmet need for the development of novel systemic therapies that achieve improvement in the three efficacy endpoints of progression-free survival (PFS), objective response rate, and overall survival in the treatment of pancreatic ductal adenocarcinoma.

### INCORPORATION BY REFERENCE

Patent documents Ser. No. 13/554,954; Ser. No. 13/595,936; Ser. No. 13/714,875; Ser. No. 13/950,111; Ser. No. 14/712,731; Ser. No. 14/650,852; Ser. No. 14/650,854; Ser. No. 14/910,565; US2011/022125; US2013/056435; US2012/069841; US2013/074809; US2013/074786; US2013/074796; US2015/0315553 are herein specifically incorporated by reference for all teachings.

### DISCLOSURE OF THE INVENTION

In one aspect, the present invention provides methods for the treatment of pancreatic ductal adenocarcinoma in a human patient, comprising the administration of a soluble AXL variant polypeptide as a first-line therapy, according to a regimen determined to achieve stable disease/response (e.g., overall response rate (ORR)), longer progression free survival (PFS), and overall survival (OS) as compared to control.

In another aspect, the present invention provides methods for the treatment of pancreatic ductal adenocarcinoma in a human patient, comprising the administration of a soluble AXL variant polypeptide in combination with nab-paclitaxel and gemcitabine as first-line therapy according to a regimen determined to achieve stable disease/response (e.g., overall response rate (ORR)), longer PFS, and OS as compared to control. In some embodiments, the soluble AXL variant polypeptide may offer additive or synergistic benefit to the therapeutic activity of nab-paclitaxel and/or gemcitabine.

In some embodiments, the soluble AXL polypeptide is a soluble AXL variant polypeptide, wherein said soluble AXL variant polypeptide lacks the AXL transmembrane domain, lacks a functional fibronectin (FN) domain, has one or more Ig1 domain, has one or more Ig2 domain, and wherein said AXL variant polypeptide exhibits increased affinity of the AXL variant polypeptide binding to GAS6 compared to wild-type AXL.

In some embodiments, the soluble AXL polypeptide is a soluble AXL variant polypeptide, wherein said soluble AXL variant polypeptide lacks the AXL transmembrane domain, lacks a functional fibronectin (FN) domain, has one Ig1 domain, lacks a functional Ig2 domain and wherein said AXL variant polypeptide exhibits increased affinity of the AXL variant polypeptide binding to GAS6 compared to wild-type AXL.

In some embodiments, the AXL variant polypeptide is a fusion protein comprising an Fc domain. In some embodiments, the variant polypeptide lacks the AXL intracellular domain. In some embodiments, the soluble AXL variant polypeptide further lacks a functional fibronectin (FN) domain and wherein said variant polypeptide exhibits increased affinity of the polypeptide binding to GAS6. In some embodiments, the soluble AXL variant polypeptide comprises at least one amino acid modification relative to the wild-type AXL sequence.

In some embodiments, the soluble AXL variant polypeptide comprises at least one amino acid modification within a region selected from the group consisting of 1) between 15-50, 2) between 60-120, and 3) between 125-135 of the wild-type AXL sequence (SEQ ID NO: 1).

In some embodiments, the soluble AXL variant polypeptide comprises at least one amino acid modification at position 19, 23, 26, 27, 32, 33, 38, 44, 61, 65, 72, 74, 78, 79, 86, 87, 88, 90, 92, 97, 98, 105, 109, 112, 113, 116, 118, or 127 of the wild-type AXL sequence (SEQ ID NO: 1) or a combination thereof.

In some embodiments, the soluble AXL variant polypeptide comprises at least one amino acid modification selected from the group consisting of 1) A19T, 2) T23M, 3) E26G, 4) E27G or E27K 5) G32S, 6) N33S, 7) T381, 8) T44A, 9) H61Y, 10) D65N, 11) A72V, 12) S74N, 13) Q78E, 14) V79M, 15) Q86R, 16) D87G, 17) D88N, 18) 190M or 190V, 19) V92A, V92G or V92D, 20) I97R, 21) T98A or T98P, 22) T105M, 23) Q109R, 24) V112A, 25) F113L, 26) H116R, 27) T118A, 28) G127R or G127E, and 29) G129E and a combination thereof.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; and (d) glycine 127.

In some embodiments, the AXL variant polypeptide comprises amino acid changes relative to the wild-type AXL sequence (SEQ ID NO: 1) at the following positions: (a) aspartic acid 87 and (b) valine 92.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; (d) glycine 127 and (e) alanine 72.

In some embodiments, the AXL variant polypeptide comprises amino acid changes relative to the wild-type AXL sequence (SEQ ID NO: 1) at the following position: alanine 72.

In some embodiments, the AXL variant polypeptide glycine 32 residue is replaced with a serine residue, aspartic acid 87 residue is replaced with a glycine residue, valine 92 residue is replaced with an alanine residue, or glycine 127 residue is replaced with an arginine residue or a combination thereof.

In some embodiments, the AXL variant polypeptide residue aspartic acid 87 residue is replaced with a glycine residue or valine 92 residue is replaced with an alanine residue or a combination thereof.

In some embodiments, the AXL variant polypeptide alanine 72 residue is replaced with a valine residue.

In some embodiments, the AXL variant polypeptide glycine 32 residue is replaced with a serine residue, aspartic acid 87 residue is replaced with a glycine residue, valine 92 residue is replaced with an alanine residue, glycine 127 residue is replaced with an arginine residue or an alanine 72 residue is replaced with a valine residue or a combination thereof.

In some embodiments, the AXL variant comprises amino acid changes relative to the wild-type AXL sequence (SEQ ID NO: 1) at the following positions: (a) glutamic acid 26; (b) valine 79; (c) valine 92; and (d) glycine 127.

In some embodiments, the AXL variant polypeptide glutamic acid 26 residue is replaced with a glycine residue, valine 79 residue is replaced with a methionine residue, valine 92 residue is replaced with an alanine residue, or glycine 127 residue is replaced with an arginine residue or a combination thereof.

In some embodiments, the AXL variant polypeptide comprises at least an amino acid region selected from the group consisting of amino acid region 19-437, 130-437, 19-132, 21-121, 26-132, 26-121 and 1-437 of the wild-type AXL polypeptide (SEQ ID NO: 1), and wherein one or more amino acid modifications occur in said amino acid region.

In some embodiments, the AXL variant polypeptide comprises amino acid changes relative to the wild-type AXL sequence (SEQ ID NO: 1) at the following positions: (a) glycine 32; (b) aspartic acid 87; (c) alanine 72; and (d) valine 92.

In some embodiments, the AXL variant polypeptide glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL variant polypeptide is a fusion protein further comprising an Fc domain and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL variant polypeptide is a fusion protein comprising an Fc domain and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL variant polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant polypeptide is a fusion protein comprising an Fc domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant is a fusion protein comprising an Fc domain, lacks a functional FN domain, and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL variant polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72 and (d) valine 92.

In some embodiments, the soluble AXL variant is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, and valine 92 is replaced with an alanine residue or a combination thereof.

In some embodiments, the soluble AXL variant polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL variant polypeptide has an affinity of at least about 1×10⁻⁸M, 1×10⁻⁹M, 1×10⁻¹⁰M, 1×10⁻¹¹M or 1×10⁻¹²M for GAS6.

In some embodiments, the soluble AXL variant polypeptide exhibits an affinity to GAS6 that is at least about 5-fold stronger, at least about 10-fold stronger or at least about 20-fold stronger than the affinity of the wild-type AXL polypeptide.

In some embodiments, the soluble AXL variant polypeptide further comprises a linker. In some embodiments, the linker comprises one or more (GLY)₄SER units. In some embodiments, the linker comprises 1, 2, 3 or 5 (GLY)₄SER units. In some embodiments, the linker comprises 1 (GLY)₄SER unit.

In some embodiments, the soluble AXL variant polypeptide is a fusion protein comprising an Fc domain, a linker, lacks a functional FN domain, and having the amino acid sequence set forth in SEQ ID NO: 2 (referred to herein as “AVB-S6-500”). AVB-S6-500 has also been referred to by Applicants in the literature as “AVB-500” and as “batiraxcept”.

In some embodiments, the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0, of about 19.5, of about 20.0, of about 20.5, of about 21.0, of about 21.5, of about 22.0, of about 22.5, of about 23.0, of about 23.5, of about 24.0, of about 24.5, of about 25.0, of about 25.5, of about 26.0, of about 26.5, of about 27.0, of about 27.5, of about 28.0, of about 28.5, of about 29.0, of about 29.5, and of about 30.0 mg/kg. In some embodiments, the soluble AXL variant polypeptide will be given as IV infusion over 30 or 60 minutes at a weekly dose of 15 mg/kg. In some embodiments, the soluble AXL variant polypeptide will be given as IV infusion over 30 or 60 minutes at a weekly dose of 10 mg/kg. In some embodiments, the soluble AXL variant polypeptide will be given as IV infusion over 30 or 60 minutes at a weekly dose of 5 mg/kg. In some embodiments, the soluble AXL variant polypeptide will be given as IV infusion over 30 or 60 minutes at a weekly dose of 2.5 mg/kg. In some embodiments, the soluble AXL variant polypeptide will be given as IV infusion over 30 or 60 minutes at a weekly dose of 1 mg/kg. In some embodiments, the soluble AXL variant polypeptide will be given as IV infusion over 30 or 60 minutes at a dose of 25 mg/kg every 14 days. In some embodiments, the soluble AXL variant polypeptide will be given as IV infusion over 30 or 60 minutes at a dose of 20 mg/kg every 14 days. In some embodiments, the soluble AXL variant polypeptide will be given as IV infusion over 30 or 60 minutes at a dose of 15 mg/kg every 14 days. In some embodiments, the soluble AXL variant polypeptide will be given as IV infusion over 30 or 60 minutes at a dose of 10 mg/kg every 14 days. In some embodiments, the soluble AXL variant polypeptide will be given as IV infusion over 30 or 60 minutes at a dose of 5 mg/kg every 14 days. In some embodiments, the soluble AXL variant polypeptide will be given as IV infusion over 30 or 60 minutes at a dose of 2.5 mg/kg every 14 days. In some embodiments, the soluble AXL variant polypeptide will be given as IV infusion over 30 or 60 minutes at a dose of 1 mg/kg every 14 days.

In some embodiments, the dose of nab-paclitaxel and gemcitabine to be co-administered to the patient along with the soluble AXL variant polypeptide is selected from the group consisting of about 25, of about 50, of about 75, of about 100, of about 125, of about 150, of about 175, of about 200, of about 225, of about 250, of about 275, of about 300, of about 325, of about 350, of about 375, of about 400, of about 425, of about 450, of about 475, of about 500 mg/kg, of about 525, of about 550, of about 575, of about 600, of about 625, of about 650, of about 675, of about 700, of about 725, of about 750, of about 775, of about 800, of about 825, of about 850, of about 875, of about 900, of about 925, of about 950, of about 975, of about 1000, of about 1025, of about 1050, of about 1075, of about 1100, of about 1125, of about 1150, of about 1175, of about 1200, of about 1225, of about 1250, of about 1275, of about 1300, of about 1325, of about 1350, of about 1375, of about 1400, of about 1425, of about 1450, of about 1475, and of about 1500 mg/m². In some embodiments, the nab-paclitaxel will be given as IV infusion over 30 or 40 minutes at a weekly dose of 125 mg/m² and gemcitabine will be given as IV infusion over 30 or 40 minutes at a weekly dose of 1000 mg/m².

### MODE(S) FOR CARRYING OUT THE INVENTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

The terms “polypeptide,” “peptide” and “protein” are used interchangeably herein to refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms “antibody” and “antibodies” are used interchangeably herein and refer to a polypeptide capable of interacting with and/or binding to another molecule, often referred to as an antigen. Antibodies can include, for example “antigen-binding polypeptides” or “target-molecule binding polypeptides.” Antigens of the present invention can include for example any polypeptides described in the present invention.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. A substantially pure polypeptide or protein will typically comprise about 50%, 60%, 70%, 80% or 90% W/W of a protein sample, more usually about 95%, and e.g., will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an a-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. All single letters used in the present invention to represent amino acids are used according to recognized amino acid symbols routinely used in the field, e.g., A means Alanine, C means Cysteine, etc. An amino acid is represented by a single letter before and after the relevant position to reflect the change from original amino acid (before the position) to changed amino acid (after position). For example, A19T means that amino acid alanine at position 19 is changed to threonine.

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. In general, the cells of interest for detection, analysis, classification, or treatment in the present application include precancerous (e.g., benign), malignant, pre-metastatic, and non-metastatic cells.

The term “primary tumor” refers to all neoplastic cell growth and proliferation, whether malignant or benign, and all pre-cancerous and cancerous cells and tissues located at the anatomical site where the autonomous, unregulated growth of the cells initiated, for example the organ of the original cancerous tumor. Primary tumors do not include metastases.

The “pathology” of cancer includes all phenomena that compromise the well-being of the patient. This includes, without limitation, abnormal or uncontrollable cell growth, primary tumor growth and formation, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor (e.g., the organ containing the primary tumor). Metastasis can also be defined as several steps of a process, such as the departure of cancer cells from an original tumor site (e.g., primary tumor site) and migration and/or invasion of cancer cells to other parts of the body.

Depending on the nature of the cancer, an appropriate patient sample is obtained. As used herein, the phrase “cancerous tissue sample” refers to any cells obtained from a cancerous tumor. In the case of solid tumors which have not metastasized (for example a primary tumor), a tissue sample from the surgically removed tumor will typically be obtained and prepared for testing by conventional techniques.

By “early-stage cancer” or “early stage tumor” is meant a cancer that is not invasive or metastatic or is classified as a Stage 0, 1, or 2 cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include bladder cancer (e.g., urothelial bladder cancer (e.g., transitional cell or urothelial carcinoma, non-muscle invasive bladder cancer, muscle-invasive bladder cancer, and metastatic bladder cancer) and non-urothelial bladder cancer), squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, as well as head and neck cancer and hematological malignancies.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, surgery alone, or combinations thereof. For ease of description and not limitation, it will be understood that the refractory tumor cells are interchangeable with resistant tumor cells. In some embodiments, the cancer is resistant to standard therapies. In some embodiments, the cancer is a chemoresistant cancer. In some embodiments, the cancer is a platinum resistant cancer.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

The term “detection” includes any means of detecting, including direct and indirect detection.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

As used herein, “treatment” is an approach for obtaining beneficial or desired clinical results. For purposes of this invention, beneficial or desired clinical results include, but are not limited to, any one or more of: alleviation of one or more symptoms; diminishment of extent of disease; preventing or delaying spread (e.g., metastasis, for example metastasis to the lung or to the lymph node) of disease; preventing or delaying recurrence of disease; stabilizing, delaying or slowing of disease progression; amelioration of the disease state; remission (whether partial or total); and improving quality of life. Also encompassed by “treatment” is a reduction of pathological consequence of a proliferative disease. The methods of the invention contemplate any one or more of these aspects of treatment.

Treating may refer to any indicia of success in the treatment or amelioration or prevention of cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions. The term “therapeutic effect” refers to the reduction, elimination, or prevention of the disease, symptoms of the disease, or side effects of the disease in the subject.

The phrase “synergistic effect” refers to the effect achieved when the active ingredients used together is greater than the sum of the effects that results from using the active ingredients separately.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5 times, 2.0 times, 2.5 times, or 3.0 times the length of the treatment duration.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “objective response rate” (ORR) refers to the sum of complete response (CR) rate and partial response (PR) rate.

As used herein, “overall survival” (OS) refers to the percentage of individuals in a group who are likely to be alive after a particular duration of time.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer, including without limitation, adenocarcinoma of the ovary or prostate, breast cancer, glioblastoma, etc., including those who have undergone or are candidates for resection (surgery) to remove cancerous tissue. Subjects may be human, but also include other mammals, particularly those mammals useful as laboratory models for human disease, e.g., mouse, rat, etc.

The term “diagnosis” is used herein to refer to the identification of a molecular or pathological state, disease or condition, such as the identification of a virus infection.

A “therapeutically effective amount” refers to the amount of a compound that, when administered to a subject for treating breast or ovarian cancer, is sufficient to affect such treatment of the cancer. The “therapeutically effective amount” may vary depending, for example, on the soluble AXL polypeptide or anti-cancer therapeutic selected, the stage of the cancer, the age, weight and/or health of the patient and the judgment of the prescribing physician. An appropriate amount in any given instance may be readily ascertained by those skilled in the art or capable of determination by routine experimentation.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).).

In the case of a cancer or a tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

“In combination with”, “combination therapy” and “combination products” refer, in certain embodiments, to the concurrent administration to a patient of a first therapeutic and the compounds as used herein. In some embodiments, the combination products are administered non-concurrently. When administered in combination, each component can be administered at the same time or sequentially in any order at different points in time. Thus, each component can be administered separately but sufficiently closely in time so as to provide the desired therapeutic effect.

“Concomitant administration” of a known cancer therapeutic drug with a pharmaceutical composition of the present invention means administration of the drug and AXL variant at such time that both the known drug and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

“Inhibitors,” “activators,” and “modulators” of AXL or its ligand GAS6 are used to refer to inhibitory, activating, or modulating molecules, respectively, identified using in vitro and in vivo assays for receptor or ligand binding or signaling, e.g., ligands, receptors, agonists, antagonists, and their homologs and mimetics. The compounds having the desired pharmacological activity may be administered in a physiologically acceptable carrier to a host to modulate AXL/GAS6 function. The therapeutic agents may be administered in a variety of ways, orally, topically, parenterally e.g. intravenous, subcutaneously, intraperitoneally, by viral infection, intravascularly, etc. Intravenous delivery is of particular interest. Depending upon the manner of introduction, the compounds may be formulated in a variety of ways. The concentration of therapeutically active compound in the formulation may vary from about 0.1-100 wt. %.

### Exemplary Embodiments

AXL, MER, Tyro3 and GAS6, as well as related pathways, have been described in WO2011/091305, as well as U.S. application Ser. Nos. 13/554,954 and 13/595,936; all of which are incorporated herein by reference in their entireties for all purposes. The AXL receptor and its activating ligand, GAS6, are important drivers of metastasis and therapeutic resistance in human cancers. This signaling axis represents an attractive target for therapeutic intervention, but the strong picomolar binding affinity (14-33 pM) between endogenous GAS6 and AXL and the promiscuity of small molecule AXL inhibitors has historically presented a barrier to specific and potent inhibition of AXL. AVB-S6-500 is a highly sensitive and specific inhibitor of AXL, with apparent affinity of 93-324 femtomolar to GAS6, which is approximately 200-fold higher affinity than wild-type (WT) AXL. AVB-S6-500 binds GAS6, the sole ligand of AXL, inhibiting its interaction with AXL, thereby dramatically reducing AXL signaled invasion and migration of highly metastatic cells in vitro and inhibiting metastatic disease in preclinical models of aggressive human cancers.

The pharmacokinetics (PK) and toxicokinetics of AVB-S6-500 and pharmacodynamics (PD) of growth arrest specific-6 protein (GAS6) have been investigated in mice (intraperitoneal [IP] and intravenous [IV] routes) and monkeys (IV route) following single and repeat dosing. The PK profile of AVB-S6-500 is compatible with target-mediated drug disposition (TMDD) with 2 parallel elimination paths: normal clearance of IgG and second order that is saturable and fits the typical 2-compartment model. At low doses in the cynomolgus monkey (below 5 mg/kg), clearance is high, and half-life is short, but at doses above 5 mg/kg, clearance is lower, half-life is longer, and volume of distribution is larger. Using the TMDD model (Dirks, N. A., Clinical Pharmacokinetics, 633-659, 2010), the human dose estimated to be efficacious may range from 1 mg/kg (to ensure GAS6 levels remain at least 90% less than baseline) to 20 mg/kg (to ensure 99% abrogation of GAS6 and allowing for a 3-fold increase in GAS6 levels in patients with cancer relative to normal levels). This model was used to select first-in-human dosing, and an external validation showing good agreement between projections and clinical observations has been published (Bonifacio, L., Clinical and Translational Science, 1-8, 2019).

In a the 3-month mouse study, twice-weekly administration of AVB-S6-500, as a slow bolus IV infusion at doses of 25, 50, and 100 mg/kg was well tolerated and resulted in a no-observed-adverse-effect-level (NOAEL) of at least 100 mg/kg/dose (200 mg/kg/week). There were no mortalities and no toxicologically significant treatment-related clinical signs or effects on body or organ weight. There were no clinical observations, no changes in urinalysis parameters, no ophthalmology signs, and no macroscopic or microscopic observations of significance related to the administration of AVB-S6-500 at doses up to the NOAEL of 200 mg/kg/week.

In the 3-month monkey study, once-weekly administration of AVB-S6-500, as a 30-minute IV infusion at doses of 50, 100, and 150 mg/kg dosed weekly, was well tolerated and resulted in a NOAEL of at least 150 mg/kg/dose. There were no mortalities; no toxicologically significant treatment-related clinical signs or effects on body weights, clinical observations, urinalysis parameters, organ weights, and ophthalmology; and no macroscopic or microscopic observations of significance related to the administration of AVB-S6-500 at doses up to 150 mg/kg/dose. There were non-dose-dependent clinical pathology changes seen and those were consistent with an immune response in monkeys to the human AVB-S6-500 protein.

AVB-S6-500 was evaluated in a single-blind, placebo-controlled, first-in-human, Phase 1 single ascending dose and repeat-dose (RD) study in healthy volunteers. Single dose cohorts of 1, 2.5, 5, and 10 mg/kg were evaluated as well as 1 RD cohort dosed with 5 mg/kg once weekly for 4 weeks. Subjects were treated with either placebo (normal saline) or AVB-S6-500 given as IV infusions over 60 minutes. AVB-S6-500 was well tolerated at all doses. There were no dose related AEs or SAEs, and a maximum tolerated dose (MTD) was not reached. Any AEs based on laboratory values being outside of normal range were transient and not dose-related.

AVB-S6-500 was evaluated in a Ph1b trial in combination with either paclitaxel or pegylated liposomal doxorubicin in subjects with platinum-resistant, recurrent ovarian cancer. Fifty-three subjects were dosed with AVB-S6-500 at doses ranging from 10 to 20 mg/kg once every 2 weeks in combination with their physician-chosen chemotherapy. No dose-limiting toxicities were observed. Additionally, review of aggregate safety data across all subjects at all doses through July 2020 demonstrated that the toxicities experienced by subjects participating in any cohort of the study were consistent with the expected toxicity profile of the individual chemotherapies and disease under study. Based on clinical data from prior studies of AVB-S6-500, toxicities observed in at least 10% of subjects included fatigue (26%), infusion reaction (21%), anemia (21%), and nausea (13%). All infusion reactions have been Grade 1 or 2, none met serious criteria, and all resolved without sequelae.

In the Ph1b ovarian cancer trial, preliminary data indicated a potential exposure-PFS response relationship, with those subjects who had higher AVB-S6-500 trough levels after the first dose of AVB-S6-500 having better clinical outcomes. Simulations of 5-25 mg/kg of AVB-S6-500 suggested that a dose of 15 mg/kg will generate exposures capturing the majority of benefit to PFS in this population. Higher doses were predicted to have similar PFS, suggesting a plateau of response with respect to exposure. All 5 subjects at 15 mg/kg had clinical benefit with 1 complete response (CR), 2 partial responses (PRs), and 2 SD. When compared in nonclinical models with other anti-AXL small molecules currently in clinical development, AVB-S6-500 has a superior antitumor efficacy while displaying no toxicity in pharmacology studies (Kariolis, M. R., The Journal of Clinical Investigation, 183-198, 2017). AVB-S6-500 causes regression of tumor cells in vivo when dosing in these models in 4-7 days after tumor inoculation and establishment of small tumors in the mouse. However, AVB-S6-500 is not directly cytotoxic in vitro and under normal physiological (nonstressed) conditions. Modulation of AXL signaling by a predecessor AXL decoy receptor protein, MYD1 Fc (AXL-S6-1 hlgG), increased expression of the epithelial marker E-cadherin, consistent with the AXL decoy protein causing a mesenchymal to epithelial phenotype transition in vivo (Id). Reversal of the mesenchymal phenotype has been reported to cause growth inhibition, suppression of spheroid forming capacity and induction of apoptosis (Azmi, A. S., BMC Systems Biology, 7:85, 2013) (Ludwig, Can Res, 1-30, 2017). This is consistent with the combination treatment studies conducted with predecessor proteins, which demonstrated a relationship between AXL signaling and the cellular response to deoxyribonucleic acid (DNA) damage in breast, pancreatic and ovarian cancer models. The damage was observed increased in combination with cytotoxic chemotherapies such as doxorubicin and gemcitabine studies (Kariolis, M. R., The Journal of Clinical Investigation, 183-198, 2017). Thus, inhibiting the AXL/GAS6 pathway in stressed cells (due to transition from mesenchymal to epithelial phenotype and/or in combination with cytotoxic agents) appears to lead to cell death in vivo.

In vivo studies have demonstrated a relationship between AXL signaling and cellular response to DNA damage in pancreatic models, and more so in combination with gemcitabine (Kariolis, M. R., The Journal of Clinical Investigation, 183-198, 2017). Thus, inhibiting the AXL/GA6 pathway in stressed cells appears to lead cell death in vivo.

In pancreatic mouse models, a decrease in metastasis development was observed with AVB-S6-500 monotherapy. Significant reductions in metastatic tumor weight and number were observed in pancreatic (PDA1-1) models when combined with gemcitabine. In addition, survival was prolonged, and fibrosis was substantially decreased, a key finding in this hard-to-treat cancer given that fibrosis likely decreases the efficacy of co-administered chemotherapeutic agents or immuno-therapeutics by inhibiting access of the drug and T-cells to the tumor cells (Provenzano, 2013).

Methods of the present invention include methods for the treatment of pancreatic ductal adenocarcinoma in a human patient, comprising the administration of a soluble AXL variant polypeptide as a first-line therapy, according to a regimen determined to achieve stable disease/response (e.g., overall response rate (ORR)), longer progression free survival (PFS), and overall survival (OS) as compared to control.

The present invention further provides methods for the treatment of pancreatic ductal adenocarcinoma in a human patient, comprising the administration of a soluble variant AXL variant polypeptide in combination with nab-paclitaxel and gemcitabine as first-line therapy according to a regimen determined to achieve stable disease/response (e.g., overall response rate (ORR)), longer PFS, and OS as compared to control. In some embodiments, the soluble AXL variant polypeptide may offer additive or synergistic benefit to the therapeutic activity of nab-paclitaxel and/or gemcitabine.

In some embodiments, the methods prolong progression free survival as compared to control. In some embodiments, the methods prolong overall survival as compared to control. In some embodiments, the methods achieve improved progression free survival as compared to control. In some embodiments, the methods achieve improved time to second subsequent therapy as compared to control. In some embodiments, the methods have been determined to not have a detrimental effect on Quality of Life as determined by FOSI and/or EQ-5D-5L.

In still some embodiments, therapeutic entities of the present invention are often administered as pharmaceutical compositions comprising an active therapeutic agent, i.e., and a variety of other pharmaceutically acceptable components. (See Remington's Pharmaceutical Science, 15.sup.th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. In some embodiments, the composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

The pharmaceutical compositions are generally formulated as sterile, substantially isotonic and in full compliance with all Good Manufacturing Practice (GMP) regulations of the U.S. Food and Drug Administration. Preferably, a therapeutically effective dose of the polypeptide compositions described herein will provide therapeutic benefit without causing substantial toxicity.

Toxicity of the proteins described herein can be determined by standard pharmaceutical procedures in cell cultures or experimental animals, e.g., by determining the LD50 (the dose lethal to 50% of the population) or the LD 100 (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

In some embodiments, the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0, of about 19.5, of about 20.0, of about 20.5, of about 21.0, of about 21.5, of about 22.0, of about 22.5, of about 23.0, of about 23.5, of about 24.0, of about 24.5, of about 25.0, of about 25.5, of about 26.0, of about 26.5, of about 27.0, of about 27.5, of about 28.0, of about 28.5, of about 29.0, of about 29.5, and of about 30.0 mg/kg. In some embodiments, the soluble AXL variant polypeptide will be given as IV infusion over 30 or 60 minutes at a weekly dose of 15 mg/kg. In some embodiments, the soluble AXL variant polypeptide will be given as IV infusion over 30 or 60 minutes at a weekly dose of 10 mg/kg. In some embodiments, the soluble AXL variant polypeptide will be given as IV infusion over 30 or 60 minutes at a weekly dose of 5 mg/kg. In some embodiments, the soluble AXL variant polypeptide will be given as IV infusion over 30 or 60 minutes at a weekly dose of 2.5 mg/kg. In some embodiments, the soluble AXL variant polypeptide will be given as IV infusion over 30 or 60 minutes at a weekly dose of 1 mg/kg. In some embodiments, the soluble AXL variant polypeptide will be given as IV infusion over 30 or 60 minutes at a dose of 25 mg/kg every 14 days. In some embodiments, the soluble AXL variant polypeptide will be given as IV infusion over 30 or 60 minutes at a dose of 20 mg/kg every 14 days. In some embodiments, the soluble

AXL variant polypeptide will be given as IV infusion over 30 or 60 minutes at a dose of 15 mg/kg every 14 days. In some embodiments, the soluble AXL variant polypeptide will be given as IV infusion over 30 or 60 minutes at a dose of 10 mg/kg every 14 days. In some embodiments, the soluble AXL variant polypeptide will be given as IV infusion over 30 or 60 minutes at a dose of 5 mg/kg every 14 days. In some embodiments, the soluble AXL variant polypeptide will be given as IV infusion over 30 or 60 minutes at a dose of 2.5 mg/kg every 14 days. In some embodiments, the soluble AXL variant polypeptide will be given as IV infusion over 30 or 60 minutes at a dose of 1 mg/kg every 14 days.

In some embodiments, the dose of nab-paclitaxel and gemcitabine to be co-administered to the patient along with the soluble AXL variant polypeptide is selected from the group consisting of about 25, of about 50, of about 75, of about 100, of about 125, of about 150, of about 175, of about 200, of about 225, of about 250, of about 275, of about 300, of about 325, of about 350, of about 375, of about 400, of about 425, of about 450, of about 475, of about 500 mg/kg, of about 525, of about 550, of about 575, of about 600, of about 625, of about 650, of about 675, of about 700, of about 725, of about 750, of about 775, of about 800, of about 825, of about 850, of about 875, of about 900, of about 925, of about 950, of about 975, of about 1000, of about 1025, of about 1050, of about 1075, of about 1100, of about 1125, of about 1150, of about 1175, of about 1200, of about 1225, of about 1250, of about 1275, of about 1300, of about 1325, of about 1350, of about 1375, of about 1400, of about 1425, of about 1450, of about 1475, and of about 1500 mg/m². In some embodiments, the nab-paclitaxel will be given as IV infusion over 30 or 40 minutes at a weekly dose of 125 mg/m² and gemcitabine will be given as IV infusion over 30 or 40 minutes at a weekly dose of 1000 mg/m².

### Example 1

### A Phase 1b/2 Randomized Study of AVB-S6-500 plus Nab-paclitaxel and Gemcitabine in Patients with Locally Advanced or Metastatic Pancreatic Adenocarcinoma

AVB-S6-500 solution (20 mg/mL AVB-S6-500, 0.01% polysorbate 80, 10% mono-, di-sodium phosphate, 9% sucrose, pH 7.0) for infusion will be packaged and labeled according to current Good Manufacturing Practices and supplied to the clinical site in 20 ml vials containing 10 mL sterile solution [total AVB-S6-500 content is 200 mg per vial]. The AVB-S6-500 volume is adjusted according to the subject's weight and diluted prior to infusion.

On days when all three drugs are administered, the sequence of administration is AVB-S6-500 infusion first followed by at least a 30-minute observation period, then nab-paclitaxel immediately followed by gemcitabine. AVB-S6-500 (15 mg/kg) will be administered by a 1-hour IV infusion on Days 1 and 15 of each 28-day cycle. Subjects will receive premedication with anti-H1 antagonists (anti-H1) and anti-H2 antagonists (anti-H2) (steroids optional) prior to administration of AVB-S6-500 to reduce the risk and severity of infusion reactions. Nab-paclitaxel (125 mg/m2) will be administered by a 30-40-minute IV infusion on Days 1, 8, and 15 of each 28-day cycle. Immediately following nab-paclitaxel, gemcitabine (1000 mg/m2) will be administered by a 30-minute IV infusion on Days 1, 8, and 15 of each 28-day cycle. Appropriate premedication with corticosteroids, diphenhydramine, and H2 antagonists (as per institutional practice, with IV antihistamines and steroids strongly recommended) should be administered prior to administration of nab-paclitaxel and gemcitabine. This combination is standard of care in the treatment of 1L pancreatic adenocarcinoma and will be sourced from standard commercial sources

### Phase 1b

The Ph1b portion of this study is a multicenter, open-label, single treatment arm group design to evaluate the safety, tolerability, and preliminary efficacy of AVB-S6-500 in combination with nab-paclitaxel and gemcitabine in subjects with locally advanced, recurrent, or metastatic 1L pancreatic adenocarcinoma. A safety review of the first 6 subjects who complete Cycle 1 will be conducted. If safety criteria are acceptable in these 6 subjects, the Ph1b cohort will enroll up to approximately 20 subjects. Subjects will be age 18 years or older having histologically or cytologically confirmed pancreatic adenocarcinoma. Exclusion criteria includes, among other things, prior treatment with nab-paclitaxel or gemcitabine, or concurrent treatment with any other investigational drug; Islet-cell neoplasms; having received last dose of chemotherapy (neoadjuvant or adjuvant), surgery, or radiation treatment with curative intent within 6 months prior to Cycle 1 Day 1; prior malignancy in the prior 3 years, except basal or squamous cell skin cancer, superficial bladder cancer, or carcinoma in situ of the prostate, cervix, or breast; and prior participation in a study with AVB-S6-500.

The primary objectives of Ph1b are to evaluate the safety and tolerability, and preliminary efficacy, as determined by the Investigator-assessed confirmed and unconfirmed ORR, of AVBS6-500 in combination with nab-paclitaxel and gemcitabine in subjects with locally advanced, recurrent, or metastatic 1L pancreatic adenocarcinoma. The secondary objectives of the Phase 1b study are: 1) to evaluate the PFS and DOR by investigator assessment; 2) to evaluate the pharmacokinetic (PK) profile of AVB-S6-500; 3) to evaluate pharmacodynamic GAS6 serum levels before and during treatment; and 4) to evaluate potential immunogenicity of AVB-S6-500. The exploratory objectives of Ph1b are: to evaluate efficacy by CA 19-9 status; to evaluate the relationship between tumor AXL and/or GAS6 status and clinical response or correlation with antitumor activity of AVB-S6-500; to evaluate pretreatment serum sAXL/GAS6 ratio and other mathematical transformations of pretreatment sAXL/GAS6; and evaluate pretreatment IHC levels and potentially other related proteins.

Pretreatment serum and IHC (when available) for GAS6, AXL levels, and potentially other related biomarkers will be explored for their relationships with the primary and secondary efficacy endpoints. The exploration will include employing the biomarkers as potential explanatory variables in Cox proportional hazards regression modelling of the (PFS and OS responses). Blood samples (serum) for analysis of AVB-S6-500 concentration, sAXL, GAS6, and biomarker assessments, will be collected from subjects at the following time points relative to dosing: day 1 and day 15 of cycle 1, and day 1, end of treatment, and 30 day follow-up for each subsequent cycle. At each time point, blood samples (8 mL) will be collected into serum separator tubes and processed as described in the laboratory manual. One 8 mL sample will be sufficient to analyze PK, PD, ADA, and serum biomarkers.

Subjects will continue treatment until radiological disease progression, clinical deterioration, informed consent withdrawal, death, or unacceptable toxicity. If treatment with nab-paclitaxel and gemcitabine is stopped, AVB-S6-500 as a single agent may be continued until disease progression, clinical deterioration, informed consent withdrawal, death, or unacceptable toxicity. The duration of nab-paclitaxel and gemcitabine treatment will be at the discretion of the Investigator. All subjects will be followed for OS until withdrawal of informed consent or until the end of the Survival Follow-up period up to 3 years.

The safety and tolerability of AVB-S6-500 in combination with nab-paclitaxel and gemcitabine will be evaluated in the first 6 subjects who complete at least Cycle 1 and will be evaluated after 20 subjects complete Cycle 1. In the event two or more subjects in the first 6 subjects experience the following AE, the study will be terminated. If the AE rate in the first 20 subjects is above the background toxicity rate for the nab-paclitaxel and gemcitabine combination, then it will be determined if a lower dose of AVB-S6-500 should be evaluated for this study.

As depicted in Table 1, there were no AVB-S6-500 related deaths (N=21), there were no patients with Grade 4 or 5 adverse effects, there were 6 patients with Grade 3 adverse events, 3 patients with infusion related reactions, and 2 patients had discontinuation of AVB-S6-500 due to fatigue and sepsis. There were 2 patients with Grade 4 adverse effects in the nab-paclitaxel and gemcitabine group.

TABLE 1

| | All P1b Patients |
|---|---|
| | 15 mb AVB-S6-500 + nab-paclitaxel |
| SAFETY | and gemcitabine (N = 21) |
| AVB-S6-500-Related Deaths | 0 |
| Grade 3 Adverse Effects Related | 6 (29%) Events: Back Pain, |
| to AVB-S6-500 | Dizziness, Fatigue, Muscular |
| | Weakness. Neutropenia |
| AEs Leading to AVB-S6-500 | 2 (10%) Events: Fatigue, Sepsis |
| Treatment Discontinuation | |
| AEs of Special Interest | 3 (14.3%) |
| (Infusion Related Reactions) | |

The preliminary anti-tumor activity is depicted in Table 2. At 3 months, the combination therapy provides improved efficacy over current standard of care in PDAC.

TABLE 2

| | All P1b Patients |
|---|---|
| | 15 mb AVB-S6-500 + nab-paclitaxel and |
| Best Response | gemcitabine (N = 21) |
| Confirmed Partial Response | 6 (29%) |
| Stable Disease | 5 (24%) |
| Progressive Disease | 5 (24%) |
| Not Evaluable | 3 (14%) |
| Not Applicable | 2 (10%) |

There are 4 patients with responses still on treatment at 5.4, 7.3, 7.4, and 9.2-months progression-free.

The pre-treatment serum sAXL/GAS6 biomarker analysis is depicted in Table 3. PDAC currently limited actionable biomarkers and the Table 3 data suggests that sAXL/GAS6 may be a valuable biomarker for targeting and addressing more PDAC patients.

TABLE 3

| | All P1b Patients |
|---|---|
| | 15 mb AVB-S6-500 + nab-paclitaxel |
| | and gemcitabine (N = 21) |
| PR for Patients with low | 0/6 (0%) |
| SAXL/GAS6 | |
| Confirmed PR for Patients | 6/15 (40%) |
| with high SAXL/GAS6 | |

### Phase 2

The Ph2 portion of the study will be initiated upon evidence of clinical activity from the Ph1b and a tolerable safety profile for the combination of AVB-S6-500, nab-paclitaxel, and gemcitabine. The Ph2 portion of this study is a multicenter, randomized, open-label, 2-arm design to compare the efficacy of AVB-S6-500 in combination with nab-paclitaxel and gemcitabine versus nab-paclitaxel and gemcitabine in subjects with locally advanced, recurrent, or metastatic 1L pancreatic adenocarcinoma. Approximately 60 subjects will be enrolled and randomized 1:1 into one of the two treatment arm groups: Arm A (AVB-S6-500 plus nab-paclitaxel and gemcitabine, n=30); Arm B (nab-paclitaxel and gemcitabine, n=30). Randomization will be stratified by locally advanced vs. metastatic disease at screening.

The primary objective of Ph2 is to evaluate the efficacy, as determined by Investigator-assessed PFS, of AVB-S6-500 in combination with nab-paclitaxel and gemcitabine in subjects with locally advanced, recurrent, or metastatic 1L pancreatic adenocarcinoma. The secondary objectives of the Phase 2 study are: 1) to evaluate additional efficacy endpoints (e.g., ORR, DOR, DCR, OS); 2) to evaluate the safety and tolerability of AVB-S6-500; 3) to evaluate the PK and PD profile of AVB-S6-500; and 4) to evaluate the immunogenicity of AVB-S6-500. The exploratory objectives of Ph1b are: to evaluate efficacy by CA 19-9 status; to evaluate the relationship between tumor AXL and/or GAS6 status and clinical response or correlation with antitumor activity of AVB-S6-500; to evaluate pretreatment serum sAXL/GAS6 ratio and other mathematical transformations of pretreatment sAXL/GAS6; and evaluate pretreatment IHC levels and potentially other related proteins.

AVB-S6-500 (15 mg/kg) will be administered on Days 1 and 15 and nab-paclitaxel and gemcitabine will be administered on Days 1, 8, and 15 of each 28-day cycle. The first dose of AVB-S6-500 on Cycle 1 Day 1 must be administered within 3 days of randomization. Subjects will continue treatment until radiological disease progression, clinical deterioration, informed consent withdrawal, death, or unacceptable toxicity. If treatment with nab-paclitaxel and gemcitabine is stopped, AVB-S6-500 as a single agent may be continued until disease progression, clinical deterioration, informed consent withdrawal, death, or unacceptable toxicity. The duration of nab-paclitaxel and gemcitabine treatment will be at the discretion of the Investigator. Any subject who discontinues for reasons other than objective radiological progression should continue to undergo scheduled objective tumor assessments until radiological progression has been observed. All subjects will be followed for OS until withdrawal of informed consent or until the end of the Survival Follow-up period up to 3 years.

Imaging assessments should be obtained at Screening, every 8 weeks (±7 days) from Cycle 1 Day 1 in Ph1b or from the date of randomization in Ph2 for the first 12 months, then every 12 weeks (±7 days) thereafter regardless of visit delays until radiologic disease progression is documented or the subject starts a new anticancer therapy. The timing for imaging studies should follow calendar days and will not be adjusted for cycle delays. The same assessment modality and technique for Screening and on-study assessments should be used throughout the study. CT of the chest, abdomen, and pelvis with contrast (or MRI in case of contrast allergy) is preferred. At Screening, images of the chest, abdomen, and pelvis are required. Additional imaging of anatomic areas with tumor involvement may be obtained as clinically indicated. For later time points, chest, abdomen, pelvis, and areas of tumor involvement should be followed throughout the study. Disease assessments will be based on RECIST v1.1 by Investigator assessment.

The primary efficacy endpoint of the study is PFS defined as the time interval between the first dose of AVB-S6-500 in Ph1b or date of randomization in Ph2, and radiologically documented disease progression or death, whichever comes first. This endpoint will be assessed by Investigator per RECIST v1.1.

OS is defined as the time interval between the first dose of AVB-S6-500 and death from any cause. Subjects who start any new anticancer therapy will continue to be followed for OS. Survival status will be collected at 12-week intervals (+2 weeks) for up to 3 years after EOT.

Investigator-assessed ORR per RECIST v1.1 is defined as the proportion of subjects who have a PR or CR response. For unconfirmed ORR (Phase 1b analysis), no confirming scan is required. For confirmed ORR (Phase 2 analysis), the confirming scan can be no earlier than 4 weeks from the first scan demonstrating response.

Evaluation of the DOR will include subjects with a confirmed CR or PR (by Investigator per RECIST v1.1) measured from the date of first response until the cancer progresses or subject death. DOR will be missing for subjects without any confirmed CR or PR.

The DCR is defined as the proportion of subjects who have a disease response of confirmed CR or PR, or SD≥16 weeks, by Investigator per RECIST v1.1.

In Ph1b, PFS and OS will be calculated in days as the date of event/censoring minus the date of first dose+1. In Ph2, PFS and OS will be calculated in days as the date of event/censoring minus the date of randomization+1. For both phases, DOR will be calculated in days as the date of PFS event/censoring minus the date of first CR or PR response+1. For PFS, OS, and DOR, the duration value will be converted to months units by dividing the duration in days by 30.4375.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize or be able to ascertain using no more than routine experimentation, many equivalents to the specific embodiments of the invention described herein. Such equivalents are intended to be encompassed by the appended claims.

1-10. (canceled)

11. A method for treating pancreatic ductal adenocarcinoma in a patient, comprising the administration of a soluble AXL variant polypeptide as first-line therapy according to a regimen determined to achieve stable disease as compared to a patient administered nab-paclitaxel in combination with gemcitabine.

12. A method according to claim 11, wherein the soluble AXL variant polypeptide lacks the AXL transmembrane domain; lacks a functional fibronectin (FN) domain; has one or more than one Ig1 domain and, optionally, one or more than one Ig2 domain; and has a set of amino acid modifications of the wild-type AXL sequence (SEQ ID NO:1), selected from the group consisting of:
1) Gly32Ser, Asp87Gly, Val92Ala, and Gly127Arg,
2) Glu26Gly, Val79Met, Val92Ala, and Gly127Glu; and
3) Gly32Ser, Ala72Val, Asp87Gly, Val92Ala, and Gly127Arg;
wherein said modification increases the affinity of the AXL polypeptide binding to Growth arrest-specific protein 6 (GAS6).

13. A method according to claim 11, wherein the soluble AXL variant polypeptide is fused to an Fc region to prepare a soluble AXL variant polypeptide fusion protein.

14. A method according to claim 13, wherein the soluble AXL variant polypeptide fusion protein comprises the amino acid sequence set forth in SEQ ID NO: 2.

15. A method according to claim 11, wherein the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0 mg/kg, of about 19.5, of about 20.0 mg/kg, of about 25.0 mg/, and of about 30.0 mg/kg.

16. A method for treating pancreatic ductal adenocarcinoma in a patient, comprising the administration of a soluble AXL variant polypeptide in combination with nab-paclitaxel and gemcitabine as first-line therapy according to a regimen determined to achieve stable disease as compared to a patient administered nab-paclitaxel in combination with gemcitabine.

17. The method according to claim 16, wherein the effects of the soluble AXL variant polypeptide in combination with nab-paclitaxel and gemcitabine are synergistic.

18. A method according to claim 16, wherein the soluble AXL variant polypeptide lacks the AXL transmembrane domain; lacks a functional fibronectin (FN) domain; has one or more than one Ig1 domain and, optionally, one or more than one Ig2 domain; and has a set of amino acid modifications of the wild-type AXL sequence (SEQ ID NO:1), selected from the group consisting of:
1) Gly32Ser, Asp87Gly, Val92Ala, and Gly127Arg,
2) Glu26Gly, Val79Met, Val92Ala, and Gly127Glu; and
3) Gly32Ser, Ala72Val, Asp87Gly, Val92Ala, and Gly127Arg;
wherein said modification increases the affinity of the AXL polypeptide binding to Growth arrest-specific protein 6 (GAS6).

19. A method according to claim 16, wherein the soluble AXL variant polypeptide is fused to an Fc region to prepare a soluble AXL variant polypeptide fusion protein.

20. A method according to claim 19, wherein the soluble AXL variant polypeptide fusion protein comprises the amino acid sequence set forth in SEQ ID NO: 2.

21. A method according to claim 16, wherein the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0 mg/kg, of about 19.5, of about 20.0 mg/kg, of about 25.0 mg/, and of about 30.0 mg/kg.

22. A method according to claim 16, wherein the dose of nab-paclitaxel and gemcitabine is selected from the group consisting of of about 25, of about 50, of about 75, of about 100, of about 125, of about 150, of about 175, of about 200, of about 225, of about 250, of about 275, of about 300, of about 325, of about 350, of about 375, of about 400, of about 425, of about 450, of about 475, of about 500 mg/kg, of about 525, of about 550, of about 575, of about 600, of about 625, of about 650, of about 675, of about 700, of about 725, of about 750, of about 775, of about 800, of about 825, of about 850, of about 875, of about 900, of about 925, of about 950, of about 975, of about 1000, of about 1025, of about 1050, of about 1075, of about 1100, of about 1125, of about 1150, of about 1175, of about 1200, of about 1225, of about 1250, of about 1275, of about 1300, of about 1325, of about 1350, of about 1375, of about 1400, of about 1425, of about 1450, of about 1475, and of about 1500 mg/m².

23. A method according to claim 16, wherein the dose of the soluble AXL variant polypeptide is 15 mg/kg given bi-weekly, the dose of nab-paclitaxel is 125 mg/m² weekly, and the dose of gemcitabine is 1000 mg/m² weekly.
